(19) United States
(12) Patent Application Publication
(10) Pub. No.: US 2002/0046409 A1
(43) Pub. Date: Apr. 18, 2002

(54) Roles for Nkx3.1 in prostate development and cancer

(75) Inventors: Cory Abate-Shen, Warren, NJ (US); Michael M. Shen, Warren, NJ (US); Thomas Gridley, Bar Harbor, ME (US)

Correspondence Address: Richard R. Muccino, 758 Springfield Avenue, Summit, NJ, 07901, US

(73) Assignee: University of Medicine & Dentistry of New Jersey, Newark, NJ (US)

(21) Appl. No.: 09/756,151

(22) Filed: Jan. 6, 2001

Related U.S. Application Data
(60) Provisional application No. 60/194,270, filed on Mar. 30, 2000.

(51) Int. Cl. A01K 67/027

(52) U.S. Cl. 800/3; 800/10; 800/18

(57) ABSTRACT

The present invention pertains to a mutant mouse comprising a Nkx3.1 gene having a disruption in at least one exon. The disruption has been introduced into the genome of the mouse by homologous recombination in an embryonic stem cell. The disruption provides a null mutation which results in no expression of Nkx3.1 by the mouse and in defects in prostate ductal morphogenesis and secretory protein production in the mouse. The mouse displays prostatic epithelial hyperplasia and dysplasia. The present invention pertains to a method for screening for a drug, or other therapeutic intervention, useful for preventing or treating prostate cancer. The method comprises administering a drug, or other therapeutic intervention, to a mutant mouse predisposed towards prostate cancer; diagnosing the mutant mouse for prostate a cancer precursor; and comparing the mutant mouse with a control mutant mouse not treated with the drug, or other therapeutic intervention. The presence of a prostate cancer precursor in the mutant mouse in an amount lower than the amount in the control mouse is indicative of a drug, or other therapeutic intervention, useful for preventing or treating prostate cancer.

### BACKGROUND OF THE INVENTION

This application claims priority from provisional patent application no. 60/194,270, filed Mar. 30, 2000.

### GOVERNMENT LICENSING RIGHTS

The experiments in this application were supported by National Institutes of Health Grant Number CA76501, United States Army Prostate Cancer Research Program Grant Number DAMD17-98-1-8532, National Institutes of Health Grants Numbers NS36437 and HD34883; National Institutes of Health Grant Number CA34196; and National Institutes of Health Training Grant Number T32-MH019957. The United States Government has a paid-up license in this invention and the right in limited circumstances to require the patent owner to license others on reasonable terms as provided by the terms of the Grants awarded by the National Institutes of Healths and the United States Army Prostate Cancer Research Program.

1. Field of the Invention

The present invention pertains to a mutant mouse comprising a Nkx3.1 gene having a disruption in at least one exon. The disruption has been introduced into the genome of the mouse by homologous recombination in an embryonic stem cell. The disruption provides a null mutation which results in no expression of Nk3.1 by the mouse and in defects in prostate ductal morphogenesis and secretory protein production in the mouse. The mouse displays prostatic epithelial hyperplasia and dysplasia. The present invention pertains to a method for screening for a drug, or other therapeutic intervention, useful for preventing or treating prostate cancer. The method comprises administering a drug, or other therapeutic intervention, to a mutant mouse predisposed towards prostate cancer; diagnosing the mutant mouse for prostate a cancer precursor; and comparing the mutant mouse with a control mutant mouse not treated with the drug, or other therapeutic intervention. The presence of a prostate cancer precursor in the mutant mouse in an amount lower than the amount in the control mouse is indicative of a drug, or other therapeutic intervention, useful for preventing or treating prostate cancer.

2. Description of the Background

The disclosures referred to herein to illustrate the background of the invention and to provide additional detail with respect to its practice are incorporated herein by reference and, for convenience, are referenced in the following text and respectively grouped in the appended bibliography.

Genetically altered mice are valuable research tools for the biotechnology and pharmaceutical industries and for academic scientists because they serve as mammalian models of human disease. Mice that are missing a known gene can provide important insights into the function of a gene in a living animal, thereby confirming theories about the role of the gene. Transgenic mice can be used commercially to validate drug targets by helping researchers determine whether a specific gene is involved in a disease. The researchers can then determine whether the gene (or its protein) is a good target for their drug-screening efforts. Transgenic mice can be used as a means of obtaining critical pre-clinical information about the efficacy and toxicity of candidate pharmaceutical compounds.

A transgenic mouse is a mouse that has a foreign gene added to all of its cells. A knock-out mouse is a mouse that has had a specific gene deleted (or made inactive) in all of its cells. The making of a transgenic or knock-out mouse is a long and laborious process. First, the genetic change is engineered in a single mouse embryonic stem cell, an undifferentiated cell that has the potential to turn into any cell in the body. The altered stem cell is then added to an early-stage mouse embryo that is implanted into a surrogate mother. The researchers will then breed the progeny of these mice for several generations to obtain mice that have the genetic alteration in all of their cell.

The prostate gland is of paramount importance for human disease, due to the increasing incidence of benign prostatic hyperplasia and prostate carcinoma in aging men. In fact, prostate carcinoma now represents the second leading cause of cancer death in American men (Coffey 1992; Landis et al. 1998). Nonetheless, little is known about the molecular factors that contribute to the onset or progression of prostate cancer. A primary impediment for identifying relevant molecular factors has been the paucity of information regarding the mechanisms of normal prostate growth and differentiation. Indeed, few regulatory genes are known to be expressed specifically during prostate development, or to be required for prostate function.

The prostate is a ductal gland situated at the base of the bladder that contributes secretory proteins to the seminal fluid. At maturity, the prostate is comprised of tall columnar epithelium surrounded by smooth muscle stroma (Cunha et al. 1987; Cunha 1994). Signaling interactions between epithelium and mesenchyme are required for normal prostate growth and differentiation, while deranged interactions may contribute to the inappropriate re-activation of cellular proliferation that occurs during aging (McNeal 1978; Hayward et al. 1996). During embryogenesis, inductive signals from the urogenital sinus mesenchyme induce the adjacent epithelium to form prostatic buds (Cunha et al. 1987; Cunha 1994). Postnatally, reciprocal interactions between epithelium and stroma (mesenchyme) are also required for ductal morphogenesis and prostate maturation (Donjacour and Cunha 1988). At all stages of prostate development as well as maturity, these tissue interactions require functional androgen receptors, initially in the mesenchyme and subsequently in the epithelium as well (Cunha et al. 1987; Cunha 1994). Although it is known that reciprocal signaling interactions are responsible for prostate formation and function, the relevant molecular factors are largely undefined.

Among the few regulatory genes known to be expressed in the prostate, the Nkx3.1 homeobox gene is of particular interest because it maps to the minimal region of human chromosome 8p21 (He et al. 1997; Voeller et al. 1997) that undergoes loss of heterozygosity in 60-80% of prostate tumors (Bergerheim et al. 1991; Bova et al. 1993; Trapman et al. 1994; Cher et al. 1996; Vocke et al. 1996).

### SUMMARY OF THE INVENTION

The present invention pertains to a mutant mouse comprising a Nkx3.1 gene having a disruption in at least one exon. The disruption has been introduced into the genome of the mouse by homologous recombination in an embryonic stem cell. The disruption provides a null mutation which results in no expression of Nkx3.1 by the mouse and in defects in prostate ductal morphogenesis and secretory protein production in the mouse. The mouse displays prostatic epithelial hyperplasia and dysplasia. The disruption is preferably introduced by homologous recombination using a positive-negative replacement vector to delete at least part of the coding region. The vector is preferably constructed in pPNT using a 4.1 kb EcoRI fragment as the 3′ flank and a 4.5 kb NotI-EcoRI fragment as the 5′ flank.

The present invention also pertains to a method for screening for a drug, or other therapeutic intervention, useful for preventing or treating prostate cancer which comprises the steps of:

(a) administering a drug, or other therapeutic intervention, to a mutant mouse predisposed towards prostate cancer;

(b) diagnosing the mutant mouse from step (a) for a prostate cancer precursor; and

(c) comparing the mutant mouse in step (b) with a control mutant mouse not treated with the drug, or other therapeutic intervention, from step (a); wherein the presence of a prostate cancer precursor in the mutant mouse in step (b) in an amount lower than the amount in the control mouse in step (c) is indicative of a drug, or other therapeutic intervention, useful for preventing or treating prostate cancer; and wherein the mutant mouse predisposed towards prostate cancer comprises a mouse having Nkx3.1 gene having a disruption in at least one exon, wherein the disruption has been introduced into the genome of the mouse by homologous recombination in an embryonic stem cell, the disruption providing a null mutation which results in no expression of Nkx3.1 by the mouse, in defects in prostate ductal morphogenesis and secretory protein production in the mouse, and wherein the mouse displays prostatic epithelial hyperplasia and dysplasia.

### BRIEF DESCRIPTION OF THE FIGURES

FIG. 1 illustrates prostate-specific expression of Nkx3.1 in adult male mice.

FIG. 1(A) is a diagram of the male urogenital system in adult mice, showing the embryological relationships of the tissues.

FIG. 1(B) is a diagram of the male urogenital system in a newborn mouse.

FIG. 1(C) illustrates ribonuclease protection analysis using total RNA (20 mg) from the indicated tissues of adult (8-week) male mice, using a Nkx3.1 antisense riboprobe.

FIG. 2 illustrates expression of Nkx3.1 in embryonic and neonatal prostate.

FIG. 2(A) is a diagram showing transverse planes of section through the urogenital sinus, shown in panels FIGS. 2(B)-(M).

FIGS. 2(N)-(W) show Nkx3.1 expression in isolated tissues from male mice at postnatal day 0 (P0) and 8 (P8); staining is more intense at the ends of the outgrowing prostatic ducts (arrows in O, P, and S).

FIG. 3 illustrates that Nkx3.1 marks prostate differentiation in tissue recombinants.

FIG. 3(A) shows the design of the tissue recombination assay.

FIGS. 3(B)-(E) show in situ hybridization analysis of Nkx3.1 expression in tissue recombinants harvested at 1 week.

FIGS. 3(F)-(I) show Nkx3.1 expression in tissue recombinants of UGM with wild-type BLE (WT BLE) versus UGM with BLE from Testicular-feminization mice (Tfm BLE), at 2 and 4 weeks of growth.

FIG. 4 illustrates the analysis of Nkx3.1 mutant mice.

FIGS. 4(A)-(E) are the targeted disruption of Nkx3. 1. FIG. 4(A) shows the strategy for gene disruption. FIG. 4(B) is a Southern blot analysis of genomic DNA using the 5′ flanking probe, showing recovery of wild-type (+/+), heterozygous (+/−), and homozygous (−/−) adult mice. FIG. 4(C) is a Southern blot analysis using an internal probe containing the homeobox, confirming its deletion in Nkx3.1 homozygotes.

FIG. 4(D) is a polymerase chain reaction (PCR) analysis of genomic DNA from wild-type, heterozygous, and homozygous adult mice.

FIG. 4(E) illustrates ribonuclease protection analysis of total RNA from the anterior prostates of 8-week old mice, using an Nkx3.1 antisense riboprobe corresponding to the homeobox.

FIGS. 4(F)-(H) shows the morphology of male urogenital tissues from wild-type and Nkx3.1 mutant littermates. FIG. 4(F) shows the urogenital systems from wild-type (left) and Nkx3.1 homozygote (right) at 8 weeks of age, showing positions of prostatic lobes (AP, DLP, VP), bladder (B1), ductus deferens (DD), urethra (Ure), and seminal vesicles (SV). FIG. 4(G) is a higher-power view of the mutant anterior prostate shown in E, with semi-transparent ducts (arrow). FIG. 4(H) illustrates bulbourethral glands from wild-type (left) and Akx3.1 homozygote (right) at 6 weeks of age.

FIG. 4(I) shows microdissected prostatic lobes from wild-type and Nkx3.1 homozygous mice at 12 weeks of age.

FIG. 4 J)is a bar graph showing quantitation of ductal tips, analyzed as in H.

FIG. 4(K) is a bar graph showing quantitation of the histological composition of the wild-type and Nkx3.1 mutant bulbourethral glands.

FIG. 4(L) is an analysis of secretory proteins from ventral (VP) and anterior (AP) prostatic lobes, bulbourethral gland (BUG), and seminal vesicle (SV).

FIG. 5 illustrates the histology of Nkx3.1 mutant mice.

FIGS. 5(A)-(U) show hematoxylin-eosin staining of paraffin sections of bulbourethral glands (BUG), anterior prostate (AP), and dorsolateral prostate (DLP) in wild-type (Nkx3.1^{+/+}), heterozygous (Nkx3.1^{+/-}), and homozygous (Nkx3.1^{-/-}) mice at 4, 12, and 45 weeks of age. At 12 weeks of age,

FIGS. 5 (A)-(D), the wild-type bulbourethral gland FIGS. 5(A,B) contains differentiated mucin-producing cells, while the homozygous gland (C,D) largely contains cells with ductal morphology.

FIGS. 5(V)-(X) show Ki67 immunoreactivity in the anterior prostates of wild-type (V), heterozygous (W), and homozygous (X) Nkx3.1 mice at 6 weeks of age.

FIG. 6 shows a model for Nkx3.1 activities in prostate development, maturation, and carcinogenesis.

### DETAILED DESCRIPTION OF THE INVENTION

In aging men, the prostate gland becomes hyperproliferative and displays a propensity towards carcinoma. Although this hyperproliferative process has been proposed to represent an inappropriate re-activation of an embryonic differentiation program, the regulatory genes responsible for normal prostate development and function are largely undefined. Here it is shown that the murine Nkx3.1 homeobox gene is the earliest known marker of prostate epithelium during embryogenesis, and that it is subsequently expressed at all stages of prostate differentiation in vivo as well as in tissue recombinants. A null mutation for Nkx3.1 obtained by targeted gene disruption results in defects in prostate ductal morphogenesis and secretory protein production. Notably, Nkx3.1 mutant mice display prostatic epithelial hyperplasia and dysplasia that increases in severity with age. This epithelial hyperplasia and dysplasia also occurs in heterozygous mice, indicating haploinsufficiency for this phenotype. Since human Nkx3.1 is known to map to a prostate cancer hotspot, it is proposed that Nkx3.1 is a prostate-specific tumor suppresser gene, and that loss of a single allele may predispose to prostate carcinogenesis. The Nkx3.1 mutant mice provide a unique animal model for examining the relationship between normal prostate differentiation and early stages of prostate carcinogenesis.

In accord with the present invention, a mutant mouse is provided comprising a Nkx3.1 gene having a disruption in at least one exon. The disruption has been introduced into the genome of the mouse by homologous recombination in an embryonic stem cell. The disruption provides a null mutation which results in no expression of Nkx3.1 by the mouse and in defects in prostate ductal morphogenesis and secretory protein production in the mouse. The mouse displays prostatic epithelial hyperplasia and dysplasia. The disruption is preferably introduced by homologous recombination using a positive-negative replacement vector to delete at least part of the coding region or the homeodomain, or both. The vector is preferably constructed in pPNT using a 4.1 kb EcoRl fragment as the 3′ flank and a 4.5 kb NotI-EcoRI fragment as the 5′ flank.

The present invention also pertains to a method for screening for a drug, or other therapeutic intervention, useful for preventing or treating prostate cancer. The method comprises the steps of (a) administering a drug, or other therapeutic intervention, to a mutant mouse predisposed towards prostate cancer; (b) diagnosing the mutant mouse from step (a) for a prostate cancer precursor; and (c) comparing the mutant mouse in step (b) with a control mutant mouse not treated with the drug, or other therapeutic intervention, from step (a); wherein the presence of a prostate cancer precursor in the mutant mouse in step (b) in an amount lower than the amount in the control mouse in step (c) is indicative of a drug, or other therapeutic intervention, useful for preventing or treating prostate cancer. The mutant mouse predisposed towards prostate cancer comprises a mouse having Nkx3.1 gene having a disruption in at least one exon. The disruption is introduced into the genome of the mouse by homologous recombination in an embryonic stem cell, the disruption providing a null mutation which results in no expression of Nk3.1 by the mouse, in defects in prostate ductal morphogenesis and secretory protein production in the mouse, and wherein the mouse displays prostatic epithelial hyperplasia and dysplasia. A prostate cancer precursor is a histological lesion known to precede prostate cancer. Prostate cancer precursors are also known as prostatic epithelial neoplasias.

Results

Restricted Expression of Nkx3.1 in Adult Prostate and Bulbourethral Glands

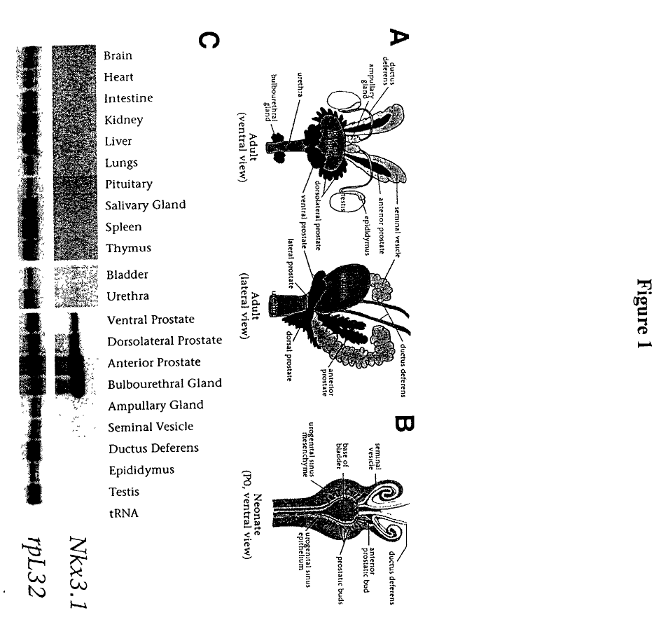

In rodents, the prostate gland consists of three lobes, the anterior prostate (AP; also known as the coagulating gland), the dorsolateral prostate (DLP), and the ventral prostate (VP) (FIG. 1A). These lobes are arranged circumferentially around the urethra and display characteristic patterns of ductal branching and protein secretion (Cunha et al. 1987). In contrast, the adult human prostate lacks discernible lobular organization, and instead completely envelops the urethra at the base of the bladder (Cunha et al. 1987). The prostatic lobes and bulbourethral gland (BUG; also known as Cowper's gland in humans) arise from the endodermally-derived urogenital sinus epithelium, while other ductal tissues of the male urogenital system arise from the mesodermally-derived Wolffian ducts (FIGS. 1A,B; (Cunha et al. 1987)).

The distribution of Nkx3. 1 transcripts in the adult mouse was examined, with particular emphasis on male urogenital tissues. It was found by ribonuclease protection analysis that Nkx3.1 expression was highly restricted to the three prostatic lobes and the bulbourethral gland (FIG. 1C). In contrast, Nkx3.1 transcripts were not detectable in the seminal vesicle, ampullary gland, ductus deferens, or epididymus, which are derivatives of the Wolffian duct, or in the bladder and urethra, which are non-ductal derivatives of the primitive urogenital sinus. Quantitation of Nkx3.1 transcripts demonstrated highest levels in the bulbourethral gland (normalized to 100%), followed in order by the anterior prostate (47%), dorsolateral prostate (26%), and the ventral prostate (9%). No expression was detected in other tissues examined, consistent with previous studies (Bieberich et al. 1996; Sciavolino et al. 1997). Thus, these data demonstrate that adult expression of ANkx3. 1 is restricted to ductal derivatives of the urogenital sinus.

Nkx3. 1 Expression Defines Early Stages of Prostate Development

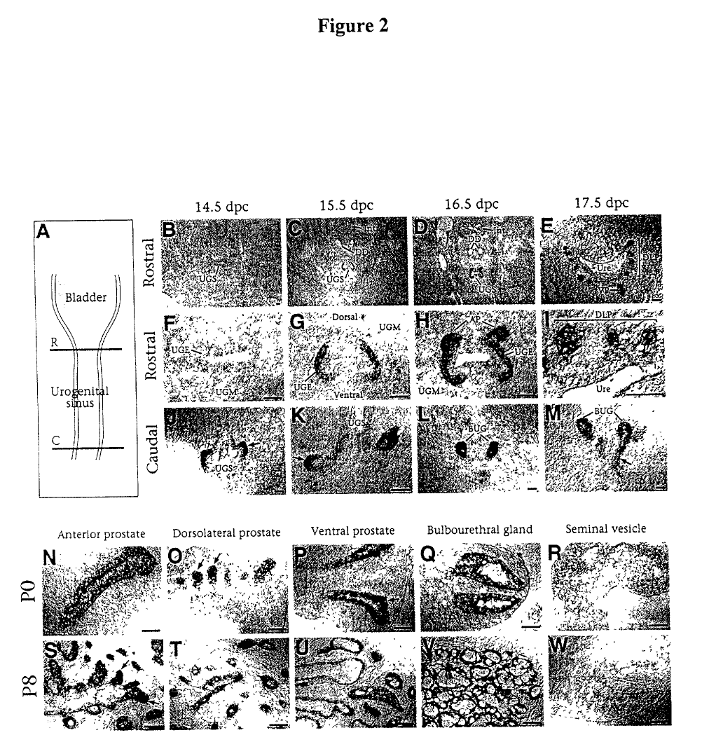

Given the highly restricted expression of Nkx3.1 in the adult prostate and bulbourethral gland, its expression was investigated during late stages of embryogenesis, when these tissues arise from the urogenital sinus. The pattern of Nkx3.1 expression by section in situ hybridization in male mouse embryos was examined from 14.5 through 17.5 days post coitum (dpc), prior to and during formation of the prostate and bulbourethral glands (FIGS. 2A-M). These results demonstrate that Nkx3.1 is the earliest known molecular marker of the prostate epithelium, and define initial steps in prostate formation.

During mid-gestation, the primitive urogenital sinus originates from the terminal hindgut through the division of the cloaca by the urorectal septum. The terminal regions of the primitive urogenital sinus form the urinary bladder and the penile urethra. The prostate and bulbourethral glands are formed from the intermediate region, which are referred to as the urogenital sinus. The prostatic lobes arise from the rostral urogenital sinus at approximately 17.5 dpc, while the bulbourethral glands arise from its caudal end at approximately 14.5 dpc.

In the rostral urogenital sinus, Nkx3.1 expression is first detected at 15.5 dpc, in a characteristic “parentheses” pattern that encompasses the lateral aspects of the urogenital sinus epithelium, and is excluded from its dorsal and ventral sides (FIGS. 2B,C,F,G). Although the urogenital sinus epithelium is multi-layered at this stage, 3.1 is only expressed in the basal layer, and not in the supra-basal layers (FIG. 2G). At 16.5 dpc, this “parentheses” pattern of expression becomes more intense at its dorsal boundaries, where the buds of the anterior prostate emerge (FIGS. 2D,H). At 17.5 dpc, Nkx3. 1 expression becomes restricted to the epithelium of the outgrowing ventral, dorsolateral, and anterior prostatic buds, and is excluded from the prospective urethral epithelium (FIGS. 2E,I). Thus, Nkx3.1 expression appears to demarcate regions where prostatic buds will arise from the urogenital sinus epithelium.

At the caudal end of the urogenital sinus, Nkx3.1 is expressed at high levels in the epithelial buds of the bulbourethral glands (FIGS. 2J-M). At 14.5 and 15.5 dpc, this expression was detected in bilateral outpouchings of the urogenital sinus epithelium into the surrounding mesenchyme (FIGS. 2J,K). At 16.5 and 17.5 dpc, Nkx3.1 continues to be expressed at high levels in the nascent bulbourethral glands, as well as in the epithelial ducts that join the glands to the prospective urethra (FIGS. 2L,M).

Nkx3.1 expression is highly restricted within the embryonic male urogenital system to the rostral and caudal ends of the urogenital sinus epithelium; transcripts were not detected at any stage in the bladder or in Wolffian duct derivatives. Furthermore, this expression pattern is male-specific, since Nkx3.1 transcripts were not detected in female urogenital sinus at any stage (data not shown). However, Nkx3.1 expression is found in several non-sexually dimorphic tissues at earlier developmental stages (Sciavolino et al. 1997; Kos et al. 1998; Treier et al. 1998).

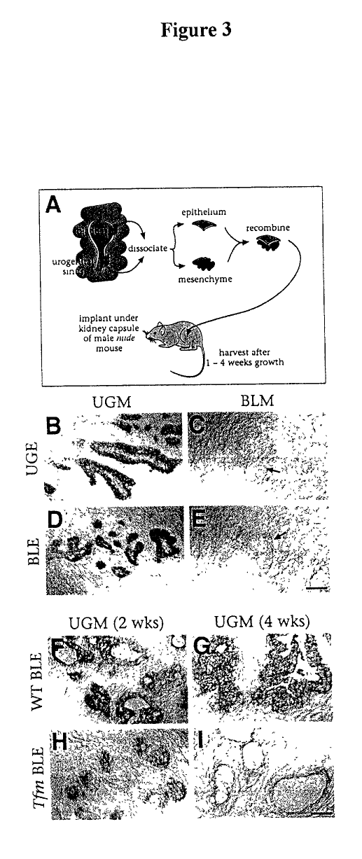

In rodents, the prostatic epithelial buds undergo extensive ductal outgrowth and branching during the first three weeks of postnatal development. Nkx3.1 expression persists at high levels in the epithelium of all three prostatic lobes at postnatal days 0, 8, and 18 (FIGS. 2N-P, S-U; data not shown). Notably, expression appears highest towards the distal ends of the outgrowing ducts, corresponding to regions of active morphogenesis (arrows in FIGS. 20, P, S). During this postnatal period, the bulbourethral glands also undergo extensive epithelial ductal branching within a capsular stromal layer (Cooke et al. 1987a; Cooke et al. 1987b). Nkx3.1 expression continues in the epithelium of the bulbourethral glands, although it appears uniform in level throughout the ducts (FIGS. 2Q,V). As is the case for embryonic development, Nkx3.1 expression is not found in other tissues of the male urogenital system (FIGS. 2R,W; data not shown). Thus, Nkx3.1 is a specific marker for ductal outgrowth and morphogenesis during postnatal growth of the prostate. Nkx3.1 marks prostate epithelium in tissue recombinants To further examine the relationship of Nkx3. 1 expression to prostate formation, a tissue recombination system was utilized (FIG. 3A). The epithelial-mesenchymal interactions required for prostate formation can be effectively recapitulated in tissue recombinants, such that appropriate combinations will give rise to prostate, identified by ductal histology and the production of characteristic secretory proteins, while different combinations will give rise to bladder or other tissues (Cunha et al. 1987; Cunha 1994). In particular, several non-prostatic epithelia (such as bladder epithelium) will form prostate when combined with the appropriate mesenchyme (such as urogenital sinus mesenchyme).

To ask whether Nkx3.1 is expressed during the acquisition of prostate identity by epithelial tissues that do not form prostate in vivo, tissue recombinations were performed with epithelial and mesenchymal components from embryonic urogenital sinus and neonatal bladder (FIGS. 3B-E). Nkx3.1 expression was only detected in tissue recombinants containing urogenital sinus mesenchyme, which induces prostate formation, but not in tissue recombinants prepared with bladder mesenchyme, which induces bladder. Nkx3.1 was expressed at early stages of prostate formation in the tissue recombinants, when the prostatic ducts have just begun to form. Importantly, Nkx3.1 expression was induced in bladder epithelium combined with urogenital sinus mesenchyme (FIG. 3D). Conversely, expression was not detectable in tissue recombinants of urogenital sinus epithelium with bladder mesenchyme (FIG. 3C), indicating that expression was lost in response to inappropriate mesenchyme. Thus, Nkx3.1 is an early and specific marker of prostate identity in tissue recombinants.

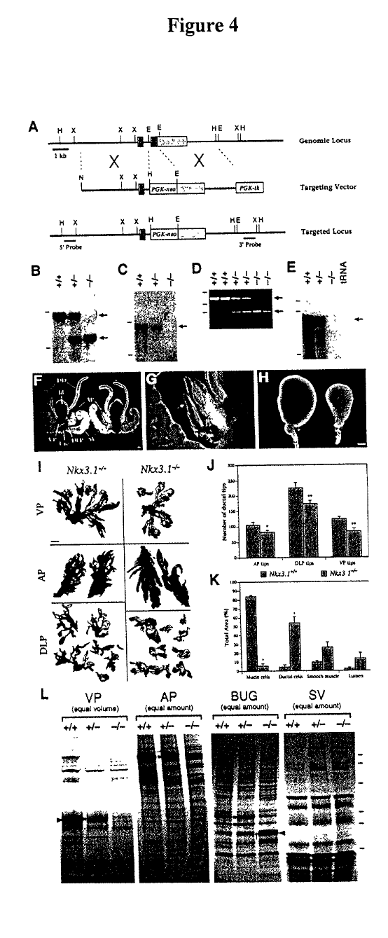

The time course of recombinant growth parallels aspects of prostate development in vivo, since tissue recombinants grown for an extended period resemble mature prostate and produce secretory proteins (Donjacour and Cunha 1993). This maturation process requires androgen receptor signaling in the epithelium (Donjacour and Cunha 1993), as shown using Testicular-feminization (Tfm) mutant mice, which lack functional androgen receptors (Lyons and Hawkes 1970). Tissue recombinants prepared with Tfm epithelium initially form prostatic-like ducts, but subsequently fail to mature and express secretory proteins. Consequently, the relationship of Nkx3.1 expression and androgen receptor signaling was examined, using prostatic tissue recombinants with normal (UGM+WT BLE) or defective (UGM+Tfm BLE) epithelial androgen receptor signaling (FIGS. 3F-I). At early stages of growth (1 and 2 weeks), Nkx3.1 expression was found in both UGM+WT BLE and UGM+Tfm BLE tissue recombinants (FIGS. 3F,H; data not shown), although at lower levels in the latter. At 4 weeks of growth, however, Nkx3.1 expression was greatly reduced or eliminated in the UGM+Tfm BLE tissue recombinants (FIGS. 3G,I). These findings indicate that epithelial androgen receptors are required for maintenance of Nkx3.1 expression, and suggest that Nkx3.1 expression is associated with mature functional prostate. Targeted disruption of Nkx3.1 results in a defect in prostate ductal morphogenesis To examine the function of Nkx3.1, targeted gene disruption was performed via homologous recombination in embryonic stem (ES) cells. A positive-negative replacement vector was constructed that would delete the homeodomain and C-terminal protein sequences, and thus should generate a null mutation (FIG. 4A). Following germline transmission of the targeted allele, heterozygous animals were intercrossed to recover viable and healthy homozygous adults that lack Nkx3.1 expression (FIGS. 4B-E). Although Nkx3.1 homozygotes are fertile, homozygous males have difficulty forming copulatory plugs with advancing age.

Analysis of homozygous mutant adult males revealed that their urogenital systems were complete, but displayed morphological defects in the prostate and bulbourethral gland (FIGS. 4F-H). Although all three prostatic lobes were present in the homozygous males, the number of prostatic ducts appeared fewer than in wild-type. Quantitative analysis of ductal tip number in adult prostatic lobes demonstrated a significant reduction to 60-75% of wild-type (FIGS. 4I,J). Moreover, this reduction in ductal tip number is evident as early as 10-11 days of age (data not shown), when ductal branching is nearly complete, but pubertal growth has not yet begun (Sugimura et al. 1986). In contrast, the overall sizes and wet weights of the prostatic lobes in the homozygotes were similar to wild-type (data not shown). Since there is reduced ductal branching without an accompanying decrease in overall size, these data indicate reduced ductal complexity in ANkx3.1 mutant prostates.

Nkx3.1 Mutant Mice Display Altered Production of Prostatic Secretory Proteins

During adult life, the primary function of the prostate is to contribute secretory proteins to the seminal fluid. In this analysis, it was observed that the anterior prostate of Nkx3.1 homozygotes frequently displayed a transparent appearance (FIG. 4G), suggesting defects in protein secretion relative to the wild-type gland, which is typically opaque. Consequently, the production of prostatic secretory proteins was examined from wild-type, heterozygous, and homozygous mutant mice by SDS-polyacrylamide gel electrophoresis (FIG. 4L).

It was found that several major prostatic secretory proteins were greatly reduced or eliminated in homozygous Nkx3.1 males (FIG. 4L, asterisks); no differences were observed in seminal vesicles used as a negative control. It was routinely observed that the pro static lobes of homozygotes contained significantly less secretory material by volume and concentration than wild-type littermate controls; for example, the total protein concentration of ventral prostate secretions in homozygotes was 2.6-fold reduced relative to wild-type (n=6). To determine the identity of a major altered protein band, microsequencing was performed on a protein that is abundant in wild-type ventral prostate secretions, but reduced or eliminated in Nkx3. 1 heterozygous and homozygous ventral prostate secretions (FIG. 4L; VP band marked with arrowhead). Sequence analyses revealed that this protein corresponds to the prostatic spermine-binding protein precursor, which is the major secretory component of the ventral prostate (Mills et al. 1987). These findings demonstrate a profound defect in the production of specific prostatic secretory proteins in Nkx3.1 mutant mice.

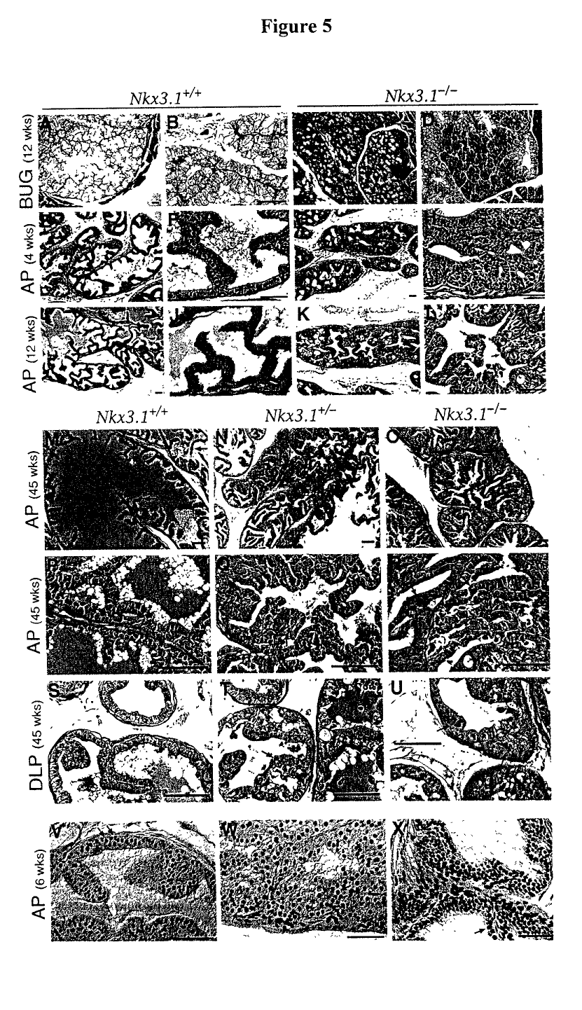

The Bulbourethral Gland of Nkx3.1 mutants displays altered cellular differentiation In Nkx3.1 mutant males, the bulbourethral glands displayed a marked reduction in overall size and cellular composition relative to wild-type controls (FIGS. 4H,K; FIGS. 5A-D). In particular, these glands were dramatically reduced in wet weight compared to wild-type (14.4±2.4 mg (n=10) versus 32.2±2.1 mg (n=6)). Furthermore, whereas the wild-type (and heterozygous) bulbourethral glands are primarily composed of mucin-producing cells, the homozygous mutant glands show a dramatic loss of these cells, and are instead composed primarily of ductal cells (FIGS. 5A-D). Quantitative analysis demonstrated a 15-fold reduction of mucin cells in the homozygote relative to the wild-type, and a corresponding 11-fold increase in ductal cells (FIG. 4K). The abundant ductal cells in Nkx3.1 mutants resemble a minor constituent of the wild-type bulbourethral gland that is primarily found near the neck of the gland.

Secretory protein production was also significantly altered in the Nkx3.1 homozygous bulbourethral gland. In particular, it was observed a novel protein species in the secretions from mutant glands (FIG. 4L, dagger), as well as reduced levels of wild-type secretory proteins (FIG. 4L, asterisks). Microsequence analysis of this novel secretory protein revealed that it corresponds to p20, an abundant component of salivary gland secretion that is related to the rat common salivary protein 1 (CSP1) (Girard et al. 1993; Bekhor et al. 1994). Taken together, these observations demonstrate that Nkx3.1 is essential for the appropriate differentiation and secretory function of the bulbourethral gland, and suggest that its loss converts a mucin-producing tissue into a ductal tissue.

Nkx3.1 Homozygous and Heterozygous Mice Display Prostatic Epithelial Hyperplasia and Dysplasia

The most notable phenotype of the Nkx3.1 mutant prostatic lobes is the histological appearance of epithelial hyperplasia and dysplasia, which becomes increasingly severe with advancing age. In wild-type adult mice, the prostate contains a simple tall columnar epithelium, with each prostatic lobe displaying a characteristic histological appearance. In particular, the epithelium of the anterior prostate forms distinct papillary tufts that are apparent by 4 weeks of age (during puberty), and which continue to form throughout adult life (FIGS. 5E,F,I,J,M,P). In contrast, as early as 4 weeks of age, the anterior prostate of homozygous Nkx3.1 mutants contains a multi-layered hyperplastic epithelium with relatively normal nuclear morphology (FIGS. 5G,H). By 12 weeks of age, the anterior prostate epithelium of homozygotes also contains dysplastic regions of epithelium showing variation in nuclear size and shape as well as abnormal mitotic figures, with a corresponding loss of lumenal space and secretory material (FIGS. 5K,L). This hyperplastic growth may account for why prostatic lobes of Nkx3.1 mutants have a reduced number of ducts, yet are not reduced in wet weight (FIGS. 4I,J; data not shown).

At one year of age, which represents the oldest mice analyzed to date, the anterior prostate of homozygotes displays extensive hyperplastic epithelium with focal areas that are severely dysplastic (FIGS. 5O,R), although no overt tumors have yet been observed. Notably, a similar but less severe hyperplastic and dysplastic epithelium is observed in heterozygous Nkx3.1 mutants, indicating haploinsufficiency for this phenotype (FIGS. 5N,Q). Furthermore, at one year of age, the dorsolateral prostate of homozygotes displays mild hyperplasia and severe dysplasia (FIG. 5U); the heterozygous dorsolateral prostates are also affected, though less severely (FIG. 5T). Interestingly, no histopathological defect has yet been observed in the ventral prostate (data not shown). Analysis of cellular proliferation using an anti-Ki67 antibody in an experimental cohort at 6 weeks of age demonstrated a 5.8-fold increase in proliferating cells in the homozygous anterior prostate, and a 4.5-fold increase in the heterozygous, as compared with wild-type (FIGS. 5V-X). These data demonstrate epithelial hyperproliferation in Nkx3.1 homozygotes and heterozygotes, indicating that the observed cytological and morphological changes model a pre-neoplastic condition.

Discussion

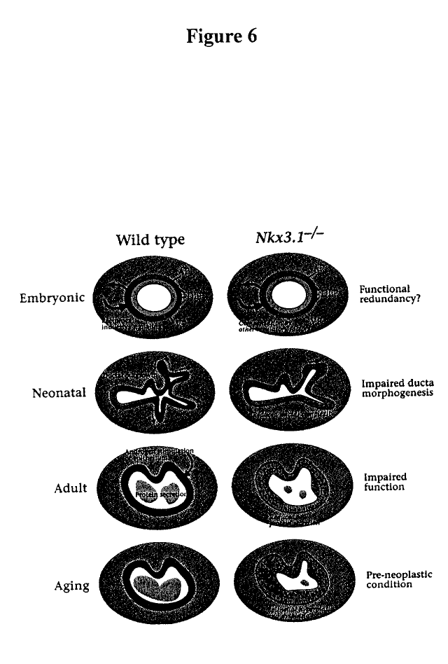

This analysis of Nkx3.1 provides a molecular link between the mechanisms that control normal prostate differentiation and those that lead to deregulated epithelial proliferation during prostate carcinogenesis. Thus, it has been shown that Nkx3. I is essential for normal morphogenesis and function of the prostate, while its inactivation leads to prostatic epithelial hyperplasia and dysplasia that model a pre-neoplastic condition (FIG. 6). Taken together with the observation that human Nkx3. 1 maps to the minimal region of chromosome 8p21 that undergoes loss of heterozygosity in prostate tumors (He et al. 1997; Voeller et al. 1997), it is proposed that Nkx3.1 maintains the differentiated state of normal prostate, while its loss represents a predisposing event for prostate carcinogenesis.

Nkx3.1 Expression Defines Early Events in Prostate Formation

Little is known about the early events of prostate formation and the molecular pathways involved in this process. Until now, it has been presumed that signals from the urogenital sinus mesenchyme are solely responsible for inducing the epithelium to form prostatic buds. However, it has been found that Nkx3.1 expression marks prospective prostate epithelium two days prior to the appearance of prostatic buds, suggesting that the urogenital sinus epithelium has a differential capacity to respond to mesenchymal signals before overt morphogenesis occurs. In particular, the “parentheses” expression pattern of Nkx3.1 defines zones of urogenital sinus epithelium, such that the dorsal boundaries correspond to the prospective anterior prostate, the intermediate regions to the dorsolateral prostate, and the ventral boundaries to the ventral prostate (compare FIG. 2G with 2H,E; FIG. 6). Thus, it is speculated that Nkx3.1 expression reveals a pre-patterning of the urogenital sinus epithelium into distinct prostatic and non-prostatic regions.

Although Nkx3.1 is the earliest known differentiation marker of the prostate epithelium, it must cooperate with other regulatory genes, since its loss of function does not result in complete failure of prostate formation (FIG. 6). Among other putative transcription factors, posterior members of the HoxD cluster are known to be expressed in adult prostate and are required for correct prostate morphogenesis (Oefelein et al. 1996; Podlasek et al. 1997). Among secreted signaling molecules, Sonic hedgehog (Shh) is known to regulate Nkx3.1 expression during somite formation (Kos et al. 1998). In preliminary studies, Shh expression has been observed in urogenital sinus epithelium prior to prostatic bud formation. This description of Nkx3.1 expression provides a foundation for future studies to identify other regulatory components responsible for prostate formation.

Roles for Nkx3. 1 in Prostate Differentiation and Function

Nkx3.1 expression is associated with all aspects of embryonic prostate development, neonatal differentiation, and adult function (FIG. 6). In many respects, the expression pattern of Nkx3.1 and the phenotype of mutant mice are analogous to those of other vertebrate Nkx homeobox genes. For example, Nkx2.5 is expressed in pre-cardiac mesoderm and in the developing heart, and null mutation results in defects in cardiac looping morphogenesis and myogenesis (Lints et al. 1993; Lyons et al. 1995). Similarly, Nkx2.1 is expressed during lung development, and targeted disruption leads to severe defects in bronchial branching (Kimura et al. 1996). These Nkx genes are expressed in highly restricted patterns during early stages of tissue specification and subsequent morphogenesis, as is observed for Nkx3.1 expression in prospective as well as differentiating prostate epithelium. Furthermore, mutations in Nkx genes result in defects in morphogenesis as well as in cellular differentiation, analogous to the defects in ductal branching and protein secretion found in Nkx3.1 mutants. Thus, like other Nkx homeobox genes, Nkx3.1 plays an essential role in organogenesis.

In addition to its role in prostate development, Nkx3.1 has a distinct and unique function in the bulbourethral gland, since Nkx3.1 mutants display a dramatic loss of mucin-producing cells and a corresponding increase of ductal cells. Despite their similar embryological origins, the prostate and bulbourethral glands are morphologically, histologically, and functionally distinct. Whereas the prostatic lobes are comprised of tall columnar epithelium surrounded by smooth muscle stroma, the bulbourethral gland primarily consists of mucin-producing cells within a skeletal muscle capsule. Notably, the epithelium of the prostate, but not that of the bulbourethral gland, is highly susceptible to hyperplastic growth and carcinogenesis. Accordingly, loss of Nkx3.1 function results in a profound alteration in cellular composition, but does not lead to hyperplastic growth of the bulbourethral epithelium.

Prostate organogenesis is intimately associated with a requirement for androgen signaling from the earliest stages of prostate formation through mature function. During embryogenesis, mesenchymal androgen receptors are required for prostate formation (Cunha et al. 1987), while during adulthood, epithelial androgen receptors are required for secretory protein production (Donjacour and Cunha 1993). These results indicate that androgen receptor signaling in the prostate epithelium is not required for the initiation of Nkx3.1 expression, since its expression precedes the appearance of functional epithelial androgen receptors (Takeda and Chang 1991). However, the absence of Nkx3.1 expression in the female urogenital system implies that mesenchymal androgen receptors are indirectly required for initiation of its expression. Furthermore, maintained expression of Nkx3.1 requires androgen receptor signaling, as shown in vivo and in cultured cells (Bieberich et al. 1996; He et al. 1997; Sciavolino et al. 1997; Prescott et al. 1998). Consistent with these observations, Nkx3.1 is expressed at early stages, but not later stages, in tissue recombinants lacking epithelial androgen receptors (UGM+Tfm BLE). These tissue recombinants do not produce secretory proteins, further underscoring the relationship between Nkx3.1 expression and secretory protein production. Since Nkx3.1 encodes a putative transcription factor, it may regulate the expression of specific secretory proteins in response to androgen receptor signaling. Potential role for Nkx3. 1 in prostate carcinogenesis In addition to its chromosomal localization to a prostate cancer “hotspot”, several lines of evidence implicate Nkx3.1 as a candidate prostate tumor suppresser gene. Notably, it is shown that Nkx3.1 mutant mice display epithelial hyperplasia and dysplasia, modeling a pre-neoplastic condition (FIG. 6). This epithelial hyperplasia and dysplasia mimics the time course of prostate cancer progression in human patients, which occurs as a consequence of aging. Furthermore, it is observed that overexpression of human or murine Nkx3.1 suppresses growth and tumorigenicity of prostate carcinoma cells in culture. At present, there is no evidence for mutations of the Nkx3.1 coding region in human prostate tumors (Voeller et al. 1997). However, this analysis of Nkx3.1 heterozygous mice demonstrates haploinsufficiency for the epithelial hyperplasia and dysplasia phenotype. Therefore, loss of a single Nkx3. 1 allele may be sufficient to promote prostate carcinogenesis in humans. Indeed, haploinsufficiency of other tumor suppresser genes has been implicated in cancer progression (Fero et al. 1998). Since candidate tumor suppresser genes are often not mutated in prostate tumor specimens, haploinsufficiency may be of general significance in prostate cancer.

While many homeobox genes have been implicated in carcinogenesis, Nkx3.1 is unusual in that it is a candidate tumor suppresser gene, rather than an oncogene. It is proposed that loss of human Nkx3.1 is an early event in prostate carcinogenesis that results in a pre-neoplastic condition, while subsequent genetic events promote progression to overt carcinoma. Candidate genetic events that may act in concert with loss of Nkx3.1 include loss of MXI1 and/or PTEN, since the corresponding mutant mice display prostatic epithelial hyperplasia and dysplasia, with no overt neoplastic transformation (Di Crisofano et al. 1998; Schreiber-Agus et al. 1998). Thus, the Nkx3.1 mutant mice should serve as an excellent model for recapitulating the molecular events of prostate cancer initiation, and for defining downstream genetic events in prostate cancer progression.

The present invention is further illustrated by the following examples which are not intended to limit the effective scope of the claims. All parts and percentages in the examples and throughout the specification and claims are by weight of the final composition unless otherwise specified.

### EXAMPLES

### Materials & Methods

Expression Analysis

Ribonuclease protection analyses were performed on total RNA isolated from individually dissected prostatic lobes or other tissues from 8-week old male virgin Swiss-Webster mice (Taconic), as described (Shen and Leder 1992). The antisense riboprobes correspond to a 286 bp cDNA fragment spanning exons 1 and 2 (FIG. 1C) or a 187 bp fragment from exon 2 that includes the homeobox (FIG. 4E). Quantitation was performed using a Phosphorlmager (Molecular Dynamics), and the Nkx3.1 signal was normalized to the L32 ribosomal protein internal control probe (Shen and Leder 1992). Note that the previously reported expression of Nkx3.1 in seminal vesicle (Sciavolino et al. 1997) was likely due to contamination by anterior prostate. For section in situ hybridization, mouse embryos were obtained at 14.5 through 17.5 days post coitum (dpc; where day 0.5 is defined as noon of the day of the copulatory plug), and sexed by PCR using primers for the Sry gene (Hogan et al. 1994). Neonatal prostatic lobes and other urogenital tissues were individually dissected at postnatal days 0, 8, and 18. In situ hybridization was carried out as described (Sciavolino et al. 1997), with at least 2 and usually 4 specimens from each stage, using a digoxigenin-labeled riboprobe corresponding to a 1 kb EcoR1 fragment of the Nkx3.1 cDNA.

For tissue recombination studies, rat urogenital sinus mesenchyme (17.5 dpc) and mouse urogenital sinus mesenchyme and epithelium (15.5 dpc) were obtained as described (Cunha and Donjacour 1987; Higgins et al. 1989). Bladder mesenchyme and epithelium was obtained (Cunha and Donjacour 1987) from adult or postnatal day 0 wild-type mice, or from homozygous Testicular-feminization (Tfm) mice (Lyons and Hawkes 1970). Tissue recombinants were grafted into adult male nude mouse hosts for one, two, or four weeks (Cunha and Donjacour 1987). Upon harvesting, tissues were processed for in situ hybridization and histology.

Gene Targeting

Nkx3.1 genomic clones were isolated from a IFIXII library constructed from 129Sv/J genomic DNA (Stratagene). The targeting vector was constructed in pPNT (Tybulewicz et al. 1991), using a 4.1 kb EcoRI fragment as the 3′ flank, and a 4.5 kb NotI-EcoRI fragment as the 5′ flank. The linearized construct was electroporated into CJ7 embryonic stem cells (Swiatek and Gridley 1993), and targeted clones were obtained at a frequency of 4% (3/85). Chimeric males obtained following blastocyst injection were bred with C57B1/6J females (Jackson Laboratories), and germline transmission was obtained from a single targeted ES clone. The targeted allele has been maintained on a hybrid 129/SvImJ and C57B1/6J . strain background, as well as on an inbred 129/SvImJ background. Results shown were obtained using mice in the hybrid background; the prostate phenotype appears similar in the 129/SvImJ inbred background.

Genotyping of the Nkx3.1 mutant mice was performed by Southern blot analysis and PCR. The sequence of the primers used for PCR analysis were: 5′ GTC TTG GAG AAG AAC TCA CCA TTG 3′ (wild-type Nkx3.1 forward); 5′ TTC CAC ATA CAC TTC ATT CTC AGT 3′ (mutated Nkx3.1 forward); and 5′ GCC AAC CTG CCT CAA TCA CTA AGG 3′ (wild-type and mutated Nkx3.1 reverse).

Analysis of the Nkx3.1 Mutant Phenotype

Analyses were performed using virgin male mice from postnatal day 0 through 12 months of age; experimental cohorts were wild-type, heterozygous, and homozygous littermates (Table 1). For analysis of wet weights and ductal tips, male reproductive organs were dissected and bilateral organ pairs weighed (Sugimura et al. 1986; Donjacour et al. 1998). The gross morphology and wet weights of the epididymus, ductus deferens, ampullary gland, seminal vesicle, preputial gland, and testis of adult homozygous mutants were identical to those of wild-type (data not shown). Prostatic ductal tips were traced and counted from digitized images. Organ weights and ductal tips were compared by Student's t-test. To determine the proportion of cell types in the bulbourethral gland, random images were captured from hematoxylin-and-eosin stained sections, and areas were calculated using NIH Image software. It is noted that the lack of morphological or histological (see below) defects in the testis or in androgen-dependent tissues such as the ductus deferens and seminal vesicle indicates that the reduced number of prostatic ductal tips is not due to decreased androgen levels; however, a very subtle defect in androgen production cannot be excluded.

For analysis of secretory proteins from dissected anterior prostate, bulbourethral gland, and seminal vesicle, secretions were collected in PBS containing 1 mM phenylmethylsulfonyl fluoride (PMSF) by gentle squeezing (Donjacour and Cunha 1993). Dorsolateral and ventral prostate secretions were recovered by scoring of the ducts, followed by centrifugation in PBS with 1 mM PMSF. Secretory proteins were resolved on 10-20% gradient SDS-PAGE gels (Bio-Rad), followed by visualization with Coomassie brilliant blue. For protein sequence analysis, individual protein bands were isolated from SDS-PAGE gels, and analysis performed at the Harvard Microchemistry Facility by microcapillary reverse-phase HPLC tandem mass spectrometry (μLC/MS/MS) on a Finnigan LCQ quadrupole ion trap mass spectrometer.

For histological analysis, dissected tissues were fixed in OmniFix 2000 (Aaron Medical Industries, St. Petersburg, Fla.), and processed for hematoxylin-and-eosin staining. For all cohorts, the prostatic lobes, seminal vesicle, ductus deferens, epididymus, and testis were examined. For one cohort (8 weeks of age), the lungs, brain, liver, kidney, heart, salivary glands, and intestines were also examined and found to have a normal histology (data not shown). The primary histological analysis was performed on a non-blinded basis independently reviewed the histological data on a blinded basis, reaching similar conclusions. Cellular proliferation was analyzed in mice at 6 and 20 weeks of age by immunohistochemical staining of formalin-fixed tissues using a rabbit polyclonal anti-Ki67 antibody (Novocastra Laboratories). Ki67-labeled nuclei were quantitated by counting approximately 3000 hematoxylin-stained nuclei from high-power microscopic fields.

Throughout this application, various publications have been referenced. The disclosures in these publications are incorporated herein by reference in order to more fully describe the state of the art.

### REFERENCES

Bekhor, I., Y. Wen, S. Shi, C. H. Hsieh, P. A. Denny, and P. C. Denny. 1994. cDNA cloning, sequencing and in situ localization of a transcript specific to both sublingual demilune cells and parotid intercalated duct cells in mouse salivary glands. Arch. Oral Biol. 39: 1011-1022.

Bergerheim, U. S. R., K. Kunimi, V. P. Collins, and P. Ekman. 1991. Deletion mapping of chromosomes 8, 10, and 16 in human prostatic carcinoma. Genes Chrom. Cancer 3: 215-220.

Bieberich, C. J., K. Fujita, W. W. He, and G. Jay. 1996. Prostate-specific and androgen-dependent expression of a novel homeobox gene. J Biol. Chem. 271: 31779-31782.

Bova, G. S., B. S. Carter, M. J. G. Bussemakers, M. Emi, Y. Fujiwara, N. Kyprianou, S. C. Jacobs, J. C. Robinson, J. I. Epstein, P. C. Walsh, and W. B. Isaacs. 1993. Homozygous deletion and frequent allelic loss of chromosome 8p22 loci in human prostate cancer. Cancer Res. 53: 3869-3873.

Cher, M. L., G. S. Bova, D. H. Moore, E. J. Small, P. R. Carroll, S. S. Pin, J. I. Epstein, W. B. Isaacs, and R. H. Jensen. 1996. Genetic alterations in untreated metastases and androgen-independent prostate cancer detected by comparative genomic hybridization and allelotyping. Cancer Res. 56: 3091-3102.

Coffey, D. S. 1992. Prostate cancer: an overview of an increasing dilemma. Cancer 71: 880-886.

Cooke, P. S., P. F. Young, and G. R. Cunha. 1987a. Androgen dependence of growth and epithelial morphogenesis in neonatal mouse bulbourethral glands. Endocrinology 121: 2153-2160.

Cooke, P. S., P. F. Young, and G. R. Cunha. 1987b. A new model system for studying androgen-induced growth and morphogenesis in vitro: the bulbourethral gland. Endocrinology 121: 2161-2170.

Cunha, G. R. 1994. Role of mesenchymal-epithelial interactions in normal and abnormal development of the mammary gland and prostate. Cancer 74: 1030-1044.

Cunha, G. R. and A. Donjacour. 1987. Mesenchymal-epithelial interactions: technical considerations. Prog. Clin. Biol. Res. 239: 273-282.

Cunha, G. R., A. A. Donjacour, P. S. Cooke, S. Mee, R. M. Bigsby, S. J. Higgins, and Y. Sugimura. 1987. The endocrinology and developmental biology of the prostate. Endocrine Rev. 8: 338-362.

Di Crisofano, A., B. Pesce, C. Cordon-Cardo, and P. P. Pandolfi. 1998. Pten is essential for embryonic development and tumour suppression. Nature Genet. 19: 348-355.

Donjacour, A. A. and G. R. Cunha. 1988. The effect of androgen deprivation on branching morphogenesis in the mouse prostate. Dev. Biol. 128: 1-14.

Donjacour, A. A. and G. R. Cunha. 1993. Assessment of prostatic protein secretion in tissue recombinants made of urogenital sinus mesenchyme and urothelium from normal or androgen-insensitive mice. Endocrinol. 132: 2342-2350.

Donjacour, A. A., A. A. Thomson, and G. R. Cunha. 1998. Enlargement of the ampullary gland and seminal vesicle but not the prostate in int2/Fgf-3 transgenic mice. Differentiation 62: 227-237.

Fero, M. L., E. Randel, K. E. Gurley, J. M. Roberts, and C. J. Kemp. 1998. The murine gene p27^{Kip1} is haplo-insufficient for tumour suppression. Nature 396: 177-180.

Girard, L. R., A. M. Castle, A. R. Hand, J. D. Castle, and L. Mirels. 1993. Characterization of common salivary protein 1, a product of rat submandibular, sublingual, and parotid glands. J. Biol. Chem. 268: 26592-26601.

Hayward, S. W., G. R. Cunha, and R. Dahiya. 1996. Normal development and carcinogenesis of the prostate. A unifying hypothesis. Ann. NY Acad. Sci. 784: 50-62.

He, W. W., P. J. Sciavolino, J. Wing, M. Augustus, P. Hudson, P. S. Meissner, R. T. Curtis, B. K. Shell, D. G. Bostwick, D. J. Tindall, E. P. Gelmann, C. Abate-Shen, and K. C. Carter. 1997. A novel human prostate-specific, androgen-regulated homeobox gene (Nkx3.1) that maps to 8p21, a region frequently deleted in prostate cancer. Genomics 43: 69-77.

Higgins, S. J., P. Young, J. R. Brody, and G. R. Cunha. 1989. Induction of functional cytodifferentiation in the epithelium of tissue recombinants. I. Homotypic seminal vesicle recombinants. Development 106: 219-234.

Hogan, B., R. Beddington, F. Costantini, and E. Lacy. 1994. Manipulating the mouse embryo. Cold Spring Harbor Laboratory Press, Cold Spring Harbor, N.Y.

Kimura, S., Y. Hara, T. Pineau, P. Fernandez-Salguero, C. H. Fox, J. M. Ward, and F. J. Gonzalez. 1996. The T/ebp null mouse: thyroid-specific enhancer-binding protein is essential for the organogenesis of the thyroid, lung, ventral forebrain, and pituitary. Genes Dev. 10: 60-69.

Kos, L., C. Chiang, and K. A. Mahon. 1998. Mediolateral patterning of somites: multiple axial signals, including Sonic hedgehog, regulate Nkx3.1 expression. Mech. Dev. 70: 25-34.

Landis, S. H., T. Murray, S. Bolden, and P. A. Wingo. 1998. Cancer statistics. CA: A Cancer Journalfor Clinicians 48: 6-29.

Lints, T. J., L. M. Parsons, L. Hartley, I. Lyons, and R. P. Harvey. 1993. Nkx-2.5: a novel murine homeobox gene expressed in early heart progenitor cells and their myogenic descendants. Development 119: 419-431.

Lyons, I., P. L. M., L. Hartley, R. Li, J. E. Andrews, L. Robb, and R. P. Harvey. 1995. Myogenic and morphogenetic defects in the heart tubes of murine embryos lacking the homeo box gene Nkx2-5. Genes Dev. 9: 1654-1666.

Lyons, M. F. and S. G. Hawkes. 1970. X-linked gene for testicular feminization in the mouse. Nature 227: 1217-1219.

McNeal, J. E. 1978. Evolution of benign prostatic enlargement . Invest. Urol. 15: 340-345.

Mills, J. S., M. Needham, T. C. Thompson, and M. G. Parker. 1987.

Androgen-regulated expression of secretory protein synthesis in mouse ventral prostate. Mol. Cell. Endocrinol. 53: 111-118.

Oefelein, M., C. Chin-Chance, and W. Bushman. 1996. Expression of the homeotic gene Hox-d13 in the developing and adult mouse prostate. J. Urol. 155: 342-346.

Podlasek, C. A., D. Duboule, and W. Bushman. 1997. Male accessory sex organ morphogenesis is altered by loss of function of Hoxd-13. Dev. Dyn. 208: 454-465.

Prescott, J. L., L. Blok, and D. J. Tindall. 1998. Isolation and androgen regulation of the human homeobox cDNA, Nkx3.1. Prostate 35: 71-80.

Schreiber-Agus, N., Y. Meng, T. Hoang, H. Hou, Jr., K. Chen, R. Greenberg, C. Cordon-Cardo, H. W. Lee, and R. A. DePinho. 1998. Role of Mxi1 in ageing organ systems and the regulation of normal and neoplastic growth. Nature 393: 483-487.

Sciavolino, P. J., E. W. Abrams, L. Yang, L. P. Austenberg, M. M. Shen, and C. Abate-Shen. 1997. Tissue-specific expression of murine Nkx3.1 in the male urogenital system. Dev. Dyn. 209: 127-138.

Shen, M. M. and P. Leder. 1992. Leukemia inhibitory factor is expressed by the preimplantation uterus and selectively blocks primitive ectoderm formation in vitro. Proc. Natl. Acad. Sci. USA 89: 8240-8244.

Sugimura, Y., G. R. Cunha, and A. A. Donjacour. 1986. Morphogenesis of ductal networks in the mouse prostate. Biol. Reprod. 34: 961-971.

Swiatek, P. J. and T. Gridley. 1993. Perinatal lethality and defects in hindbrain development in mice homozygous for a targeted mutation of the zinc finger gene Krox-20. GenesDev. 7:2071-2084.

Takeda, H. and C. Chang. 1991. Immunohistochemical and in-situ hybridization analysis of androgen receptor expression during the development of the mouse prostate gland. J. Endocrinol. 129: 83-89.

Trapman, J., H. F. Sleddens, M. M. van der Weiden, W. J. Dinjens, J. J. Konig, F. H. Schroder, P. W. Faber, and F. T. Bosman. 1994. Loss of heterozygosity of chromosome 8 microsatellite loci implicates a candidate tumor suppressor gene between the loci D8S87 and D8S133 in human prostate cancer. Cancer Res. 54: 6061-6064.

Treier, M., A. S. Gleiberman, S. M. O'Connell, D. P. Szeto, J. A. McMahon, A. P. McMahon, and M. G. Rosenfeld. 1998. Multistep signaling requirements for pituitary organogenesis in vivo. Genes Dev. 12: 1691-1704.

Tybulewicz, V. L., C. E. Crawford, P. K. Jackson, R. T. Bronson, and R. C. Mulligan. 1991. Neonatal lethality and lymphopenia in mice with a homozygous disruption of the c-abl proto-oncogene. Cell 65: 1153-1163.

Vocke, C. D., R. O. Pozzatti, D. G. Bostwick, C. D. Florence, S. B. Jennings, S. E. Strup, P. H. Duray, L. A. Liotta, M. R. Emmert-Bucke, and W. M. Lineham. 1996. Analysis of 99 microdissected prostate carcinomas reveals a high frequency of allelic loss on chromosome 8p12-21. CancerRes. 56: 2411-2416.

Voeller, H. J., M. Augustus, V. Madike, G. S. Bova, K. C. Carter, and E. P. Gelmann. 1997. Coding region of Nkx3.1, a prostate-specific homeobox gene on 8p21, is not mutated in human prostate cancers. Cancer Res. 57: 4455-4459.

TABLE 1
Number of mice analyzed

| Method | Age | +/+ | +/− | −/− |
|---|---|---|---|---|
| Prostate ductal tip counting | 1-2 days | 7 | 6 | 9 |
| | 10-12 days | 2 | 5 | 7 |
| | 12 weeks | 6 | 16 | 10 |
| Bulbourethral gland composition | 12 weeks | 5 | 0 | 5 |
| Protein secretion | 8 weeks | 4 | 1 | 4 |
| | 12 weeks | 1 | 1 | 1 |
| | 20 weeks | 1 | 0 | 1 |
| | 11-12 months | 2 | 2 | 2 |
| Histological analysis | 8 days | 1 | 1 | 1 |
| | 3-4 weeks | 2 | 3 | 2 |
| | 8 weeks | 6 | 6 | 6 |
| | 12 weeks | 4 | 6 | 4 |
| | 5 months | 1 | 2 | 2 |
| | 11-12 months | 4 | 4 | 4 |

Figure Legends

FIG. 1.

Prostate-specific expression of Nkx3.1 in adult male mice. (A) Diagram of the male urogenital system in adult mice, showing the embryological relationships of the tissues (adapted from (Cunha et al. 1987; Podlasek et al. 1997)). The anterior, dorsolateral and ventral prostatic lobes, as well as the bulbourethral glands (dark gray) are ductal derivatives of the urogenital sinus; the bladder and urethra (medium gray) are its non-ductal derivatives. The seminal vesicles, ductus deferens, epididymus, and ampullary glands (light gray) are derived from the Wolffian duct, and the testes (white) from the genital ridge. In the ventral view, only the base of the bladder is shown for clarity. (B) Diagram of the male urogenital system in a newborn mouse (postnatal day 0 (P0); adapted from (Cunha et al. 1987)). By 17.5 dpc, the prostatic buds (dark gray) arise as outbuddings of the urogenital sinus epithelium (white) into the surrounding mesenchyme (medium gray). Also shown are the Wolffian-duct-derived seminal vesicles and ductus deferens (light gray). (C) Ribonuclease protection analysis using total RNA (20 mg) from the indicated tissues of adult (8-week) male mice, using a Nkx3.1 antisense riboprobe. The rpL32 riboprobe serves as an internal control for RNA loading.

FIG. 2.

Expression of Nkx3.1 in embryonic and neonatal prostate. (A) Diagram showing transverse planes of section through the urogenital sinus, shown in panels B-M. The rostral region (R) corresponds to the location of the prospective prostatic buds, and the caudal region (C) corresponds to the prospective bulbourethral glands. (B-I) In situ hybridization analysis of Nkx3. 1 expression in transverse sections through the rostral male urogenital sinus, shown at low (B-E) and high power (F-J). (B,F) No expression is detected at 14.5 dpc. (C,G) At 15.5 dpc, Nkx3.1 expression is restricted to the lateral urogenital sinus epithelium (UGE), and is excluded from the dorsal and ventral sides (forming the “parentheses” pattern). (D,H) Nkx3.1 expression continues in the lateral UGE, with elevated expression in the emerging anterior prostatic buds. (E,) At 17.5 dpc, expression is restricted to the newly formed dorsolateral and ventral prostatic buds, and is not found in the prospective urethral epithelium. (J-M) Nkx3.1 expression in transverse sections through the caudal male urogenital sinus. (J) Expression at 14.5 dpc is found in bilateral outpouchings (arrow) from the UGE. (K-M) At 15.5 dpc through 17.5 dpc, expression is found in the nascent bulbourethral glands and the ducts (arrow in M) that join them to the prospective urethra. (N-W) Nkx3.1 expression in isolated tissues from male mice at postnatal day 0 (P0) and 8 (P8); staining is more intense at the ends of the outgrowing prostatic ducts (arrows in O, P, and S). Abbreviations: AP, anterior prostate; BUG, bulbourethral gland; C, caudal; DD, ductus deferens; DLP, dorsolateral prostate; Int, large intestine; R, rostral; UGE, urogenital sinus epithelium; UGM, urogenital sinus mesenchyme; UGS, urogenital sinus; Ure, urethra; VP, ventral prostate. Scale bar for all panels represents 50 μm.

FIG. 3.

Nkx3.1 marks prostate differentiation in tissue recombinants. (A) Design of the tissue recombination assay. Recombinants of urogenital sinus mesenchyme (UGM) with either urogenital sinus epithelium (UGE) or bl adder epithelium (BLE) form prostate, whereas recombinants of bladder mesenchyme (BLM) with either epithelium form bladder. (B-E) In situ hybridization analysis of Nkx3.1 expression in tissue recombinants harvested at 1 week. Expression is found in recombinants that form prostate (UGM+UGE and UGM+BLE), but not in those that form bladder (BLM+UGE and BLM+BLE). The arrows in C and E indicate bladder-like structures that do not express Nkx3.1. (F-I) Nkx3.1 expression in tissue recombinants of UGM with wild-type BLE (WT BLE) versus UGM with BLE from Testicular-feminization mice (Tfm BLE), at 2 and 4 weeks of growth. In B-I, scale bars represent 50 μm.

FIG. 4.

Analysis of Nkx3.1 mutant mice. (A-E) Targeted disruption of Nkx3.1. (A) Strategy for gene disruption. The Nkx3.1 locus comprises two exons (gray boxes), with the coding region (medium gray) contained in both exons, and the homeobox in the second exon (dark gray). Homologous recombination with the targeting vector deletes most of the coding region, including the homeobox. The positions of the 5′ and 3′ flanking probes used for Southern blot analysis are shown. Abbreviations: E, EcoRI; H, HindIII; N, NotI; X, XbaI. (B) Southern blot analysis of genomic DNA using the 5′ flanking probe, showing recovery of wild-type (+/+), heterozygous (+/−), and homozygous (−/−) adult mice. This probe detects a 9 kb HindIII wild-type fragment and a 6 kb fragment from the targeted allele (arrows). (C) Southern blot analysis using an internal probe containing the homeobox, confirming its deletion in Nkx3.1 homozygotes. This probe detects a 9 kb HindIII wild-type fragment (arrow), and does not hybridize to the targeted allele. Dashes in B and C indicate positions of markers at 10 and 5 kb. (D) PCR analysis of genomic DNA from wild-type, heterozygous, and homozygous adult mice. Primers (described in Materials and Methods) amplify a 707 bp fragment from wild-type genomic DNA and a 232 bp fragment from the targeted allele (arrows). Dashes indicate positions of markers at 1018, 506, and 220 bp. (E) Ribonuclease protection analysis of total RNA from the anterior prostates of 8-week old mice, using an Nkx3.1 antisense riboprobe corresponding to the homeobox. Dashes indicate positions of markers at 220, 201, and 154 nt. (F-H) Morphology of male urogenital tissues from wild-type and Nkx3.1 mutant littermates. (F) Urogenital systems from wild-type (left) and Nkx3.1 homozygote (right) at 8 weeks of age, showing positions of prostatic lobes (AP, DLP, VP), bladder (B1), ductus deferens (DD), urethra (Ure), and seminal vesicles (SV). (G) Higher-power view of the mutant anterior prostate shown in E, with semi-transparent ducts (arrow). (H) Bulbourethral glands from wild-type (7eft) and Nkx3.1 homozygote (right) at 6 weeks of age. Scale bars in F-H represent 0.5 mm. (I) Microdissected prostatic lobes from wild-type and Nkx3.1 homozygous mice at 12 weeks of age. Scale bar represents 1.0 mm. (J) Quantitation of ductal tips, analyzed as in H. The mean number of ductal tips was significantly smaller in each of the mutant prostatic lobes, at p<0.1 (*) or p<0.05 (**). (K) Quantitation of the histological composition of the wild-type and Nkx3.1 mutant bulbourethral glands. The total area analyzed was 6.1×10⁷ μm² for the wild-type glands and 2.5×10⁷ μm² for the mutant glands; significant differences from the wild-type (p<0.05) are indicated (*). In J and K, error bars represent standard error of the mean (SEM). (L) Analysis of secretory proteins from ventral (VP) and anterior (AP) prostatic lobes, bulbourethral gland (BUG), and seminal vesicle (SV). Protein secretions were collected from tissues of 8-week old male mice and resolved on a 10-20% SDS-PAGE gradient gel. Lanes labeled “equal volume” contain 4 μl of secretory material, while lanes labeled “equal amount” contain 10 mg of total protein. Asterisks (*) indicate proteins that are decreased in −/− mice, while the dagger (t) indicates a protein increased in homozygotes. Arrowheads indicate the protein bands analyzed by microsequencing. Dashes at right mark the positions of molecular weight standards at 102, 81, 46.9, 32.7, 30.2, and 24 kDa.

FIG. 5.

Histology of Nkx3.1 mutant mice. (A-U) Hematoxylin-eosin staining of paraffin sections of bulbourethral glands (BUG), anterior prostate (AP), and dorsolateral prostate (DLP) in wild-type (Nkx3.1⁺⁺), heterozygous (Nkx3.1^{+/-}), and homozygous (Nkx3.1^{-/-}) mice at 4, 12, and 45 weeks of age. (A-D) At 12 weeks of age, the wild-type bulbourethral gland (A,B) contains differentiated mucin-producing cells, while the homozygous gland (C,D) largely contains cells with ductal morphology. (E-H) At 4 weeks of age, the wild-type anterior prostate (E,F) contains immature columnar epithelial cells arranged in characteristic papillary tufts (arrow), while the homozygous anterior prostate (G,H) contains a multi-layered hyperplastic epithelium, with little lumenal space. (M-L) At 12 weeks of age, the wild-type anterior prostate (I,J) contains differentiated columnar epithelial cells with lumenal spaces filled with secretions (lightly staining eosinophilic material). The homozygous anterior prostate (KL) contains hyperplastic epithelium with mildly dysplastic regions (arrows), and little secretory material. (M-R) At 45 weeks of age, the wild-type anterior prostate (MP) contains tall columnar epithelium arranged in papillary tufts (arrow), the heterozygous anterior prostate (N,Q) contains hyperplastic epithelium with mildly dysplastic regions (arrow) and reduced lumenal space and secretory protein, and the homozygous anterior prostate (O,R) contains severely hyperplastic epithelium and regions of dysplasia (arrows). (S-U) At 45 weeks of age, the wild-type dorsolateral prostate (S) contains columnar epithelium and lumenal secretions, the heterozygous dorsolateral prostate (T) contains areas of mild dysplasia (arrow), and the homozygous dorsolateral prostate (U) contains severely dysplastic epithelium (arrows). (V-X) Ki67 immunoreactivity in the anterior prostates of wild-type (V), heterozygous (W), and homozygous (X) Nkx3.1 mice at 6 weeks of age. Arrows indicate Ki67-labeled nuclei. In total, 55 Ki67-labeled nuclei were observed out of 3767 total nuclei (1.5%) in wild-type; 207 out of 2991 (6.9%) in heterozygotes; and 315 out of 3573 (8.8%) in homozygotes). In A-L and V-X, scale bars represent 50 μm, while in M-U, scale bars represent 100 μm.

FIG. 6.

Model for Nkx3.1 activities in prostate development, maturation, and carcinogenesis. Model is described in text; expression of Nkx3.1 is shown in blue.

Throughout this disclosure, applicant will suggest various theories or mechanisms. While applicant may offer various mechanisms to explain the present invention, applicant does not wish to be bound by theory. These theories are suggested to better understand the present invention but are not intended to limit the effective scope of the claims.

While the invention has been particularly described in terms of specific embodiments, those skilled in the art will understand in view of the present disclosure that numerous variations and modifications upon the invention are now enabled, which variations and modifications are not to be regarded as a departure from the spirit and scope of the invention. Accordingly, the invention is to be broadly construed and limited only by the scope and spirit of the following claims.

We claim:

1. A mutant mouse comprising a Nkx3.1 gene having a disruption in at least one exon, wherein the disruption has been introduced into the genome of the mouse by homologous recombination in an embryonic stem cell, the disruption providing a null mutation which results in no expression of Nkx3.1 by the mouse, in defects in prostate ductal morphogenesis and secretory protein production in the mouse, and wherein the mouse displays prostatic epithelial hyperplasia and dysplasia.

2. The mouse according to claim 1, wherein the disruption by homologous recombination is made by using a positive-negative replacement vector to delete at least part of the coding region.

3. The mouse according to claim 2, wherein the vector is constructed in pPNT using a 4.1 kb EcoRI fragment as the 3′ flank and a 4.5 kb NotI-EcoRI fragment as the 5′ flank.

4. A method for screening for a drug, or other therapeutic intervention, useful for preventing or treating prostate cancer which comprises the steps of:
(a) administering a drug, or other therapeutic intervention, to a mutant mouse predisposed towards prostate cancer;
(b) diagnosing the mutant mouse from step (a) for a prostate cancer precursor; and
(c) comparing the mutant mouse in step (b) with a control mutant mouse not treated with the drug, or other therapeutic intervention, from step (a);
wherein the presence of a prostate cancer precursor in the mutant mouse in step (b) in an amount lower than the amount in the control mouse in step (c) is indicative of a drug, or other therapeutic intervention, useful for preventing or treating prostate cancer; and wherein the mutant mouse predisposed towards prostate cancer comprises a mouse having Nkx3.1 gene having a disruption in at least one exon, wherein the disruption has been introduced into the genome of the mouse by homologous recombination in an embryonic stem cell, the disruption providing a null mutation which results in no expression of Nkx3.1 by the mouse, in defects in prostate ductal morphogenesis and secretory protein production in the mouse, and wherein the mouse displays prostatic epithelial hyperplasia and dysplasia.

5. The method according to claim 4, wherein the disruption by homologous recombination is made by using a positive-negative replacement vector to delete at least part of the coding region.

6. The method according to claim 5, wherein the vector is constructed in pPNT using a 4.1 kb EcoRI fragment as the 3′ flank and a 4.5 kb NotI-EcoRI fragment as the 5′ flank.
